(19) United States
(12) Patent Application Publication
(10) Pub. No.: US 2015/0250575 A1
(43) Pub. Date: Sep. 10, 2015

(54) Surgical Mesh Joining and Fixation Using Photoactivated Collagen

(71) Applicants: Barbara Ann Soltz, Spring Valley, NY (US); Robert Soltz, Spring Valley, NY (US)

(72) Inventors: Barbara Ann Soltz, Spring Valley, NY (US); Robert Soltz, Spring Valley, NY (US)

(21) Appl. No.: 14/547,113

(22) Filed: Nov. 18, 2014

Related U.S. Application Data
(60) Provisional application No. 61/906,197, filed on Nov. 19, 2013.

(51) Int. Cl. A61F 2/00; A61L 27/50; A61L 27/34; A61B 18/20; A61B 17/064

(52) U.S. Cl. CPC A61F 2/0063; A61B 18/20; A61B 17/064; A61L 27/34; A61L 27/50; A61F 2220/005; A61F 2250/006; A61B 2017/0647; A61B 2018/00619

(57) ABSTRACT

Consistent with the present disclosure a method is provided in which a plurality of surgical meshes are provided, each of which may include a coating that has a chromophore. Alternatively, each mesh has a plurality of rivets or tacks that include the chromophore. The meshes may then be positioned to overlap one another or be provided adjacent one another inside the body cavity such that the mesh cover the wound site. The meshes may then be exposed to light at a wavelength that activates the chromophore and causes the meshes to adhere to one another and the underlying tissue. In one example, the coating or the tacks includes a combination of relatively high concentration derivatized collagen and riboflavin, lumiflavin or lumichrome.

This application claims the benefit of U.S. Provisional Application No. 61/906,197, filed on Nov. 19, 2013, the entire contents of which are incorporated herein by reference.

### BACKGROUND

A significant number of inguinal herniorrhaphies are performed annually in the United States. Related procedures such as ventral and incisional herniorrhaphy, ablation of endometriosis and other pelvic procedures account for an additional 1-2 million procedures each year. Inguinal hernia repair is the most frequent procedure and accounts for approximately $3 B in annual health care revenue. Laparoscopic approaches to these procedures have been developed but the market penetrance has been hampered for a variety of reasons, including short term and long term problems with mesh fixation, difficulties encountered during attempted peritoneal closure, adhesion formation and the potential for development of internal hernia. Laser tissue welding may provide an alternative technique to drastically improve the outcome thereby encouraging a broader clinical acceptance of laparoscopic herniorrhaphy.

Laparoscopic Hernia Repair Background—

There is broad clinical evidence supporting laparoscopic repairs are superior to open approaches because patients experience less postoperative pain, have shorter recovery time, allowing for earlier return to full activity, have a lower incidence of recurrence, a capability to perform simultaneous diagnostic laparoscopy, ligation of the hernia sac at the highest possible site and improved cosmesis. The principle of laparoscopic repair is a tensionless mesh reinforcement of the hernial defect. The primary disadvantages include the level of surgeon skill required for stable mesh fixation and peritoneal closure, a higher risk of postoperative adhesions and the need for expensive laparoscopic surgical instrumentation. However, equipment costs are offset by expenses incurred by longer patient convalescence, loss of work and the cost of disposables used in open/incisional repairs. Published analysis suggest the cost difference between laparoscopic and open approaches are minimal. Apparently the major deterrent to widespread acceptance of an laparoscopic approach is that it is technically more demanding and anatomically more challenging. Frequent laparoscopic repair approaches include:

Intraperitoneal Onlay Mesh Repair (IPOM)—

This technique involves a transabdominal examination of the myopectineal orifice and application of the prosthetic mesh directly to the peritoneal surface on the side where the hernia occurs. The herniated contents are reduced, but the peritoneum is not incised and the hernia sac is contained in place. The mesh is applied to the peritoneal surface, covering the entire myopectineal orifice and secured to underlying structures with staples or tacks. This procedure involves Cooper's ligament, iliopubic tract and transversus muscle and tendon. The advantage of this approach is simplicity and speed but the intraabdominal viscera is directly exposed to the prosthetic material. Disadvantages include reduction in graft structural integrity associated with these types of mesh and the graft may not be adequately secured with staples alone. As an alternative, sutures have been placed at three cardinal points on the graft and secured to the fascial bridge to fix the graft in place. This approach is not as popular as TAPP because surgeons are reluctant to place conventional mesh materials in the peritoneal cavity. However, the ability to cover the mesh and attach it to the peritoneal surface with a hydrophilic absorbable material is likely to facilitate IPOM repair strategies, reducing or eliminating intraabdominal adhesion formation and its attendant morbidity.

2. Laparoscopic Transabdominal Preperitoneal Repair (TAPP)—

TAPP is a widely used because of its relative ease to learn and perform. In this approach the mesh is anchored with either endohernia staples, tackers, or sutures. Because it is an intraperitoneal procedure, an incision in the peritoneum must be made to access the extraperitoneal space. Suture or staple closure and the associated healing process usually results in adhesion formation possibly creating severe complications including small bowel obstruction. Incomplete closure may result in internal herniation, causing bowel obstruction or ischemic injury to the bowel. Stapling should be done only to the superior margin of the iliopubic tract to avoid injury to femoral branches of the genitofemoral nerve. In some cases nerve paresthesia has occurred when the staples were placed low in the illiopubic tract, compressing or lacerating the genitofemoral nerve branches. The wide dissection necessary to anchor large mesh sections is limited in this approach and may therefore account for recurrence rates slightly higher than other laparoscopic procedures. Nevertheless patients who undergo this procedure are generally discharged the same day of the surgery and resume unrestricted full activity after one week.

Mesh and Mesh Fixation—

Published literature overwhelmingly support the routine use of mesh-based repairs. Surgeons now elect to use prosthetic materials for most incisional and laparoscopic procedures. Early prostheses included silver wire but later they were fabricated from synthetic materials including mono or multifilamented polypropylene (PPM), expanded polytetrafluoroethylene (ePTFE) or multifilamented polyester mesh. Mesh-like structures, woven from the suture materials at the time are formed from organic materials such as animal tendons, have been used in surgical repairs for more than a century. However, it was not until the development of synthetic polymer mesh that such techniques were widely adopted. The use of Nylon mesh for hernia repair was first described by French surgeons Acguaviva and Bourret in 1948, and was followed by the introduction of Polypropylene products in the 1960s. By the end of that decade surgeons had begun using hernia mesh for POP and SI repairs. The FDA granted approval for the first mesh specifically designed for SI repair in the 1990s, followed by a dedicated POP mesh product in 2002. Today it is estimated that in excess of 1 million meshes are inserted worldwide each year.

Meshes can be categorised in terms of weight, pore size, material, fiber type and flexibility. Heavyweight meshes tend to form a dense scar plate and are best suited to applications where mechanical stability is a factor. Lightweight meshes are formed from thin fibres and are designed to flex with normal physiological movement. They form a flexible scar and may cause less discomfort than heavyweight meshes. Fibres may be monofilament or multifilament, and the gaps between the fibres, known as pores, can vary depending on the design. In general, a smaller pore size reduces the ability of the mesh to be incorporated into the body's own tissues, which may a desirable quality if the mesh is to be used around delicate bowel tissue, to avoid unwanted adhesions. Meshes of the same material may differ between manufacturers in terms of weight, flexibility, shrinkage and potential for adhesion formation.

The ideal prosthetic material should be chemically inert, noncarcinogenic, capable of resisting mechanical stress, fabricated in any shape, sterilizable, do not excite inflammatory or foreign body reaction or induce allergic response. All of the aforementioned materials fall short of these requirements. In fact, signs of inflammatory response may persist for many years. While PPM (Marlex™ or Prolene™) and ePTFE remain the most frequently used, it is well known that PPM shrinks, contracts and stiffens over time. Most surgeons avoid exposing PPM to the peritoneal cavity because it allows ingrowth of the viscera, leading to fistulas of the gastrointestinal tract, erosion or bowel obstruction and the formation of dense adhesions. ePTFE mesh is considered safe for laparoscopic strategies with minimal capacity to form adhesions but is more difficult to manipulate (does not unfold easily), is opaque, reflecting light from the laparoscope, has low porosity, hydrophobic in nature (favors seroma formation) and has poor integration into the abdominal wall necessitating a complete fastening with sutures or staples. Polyester mesh (Mersilene™) is produced in sheet form for hernia repair and is similar to the woven Dacron used for vascular prosthetics and for the reinforcement of myocardium and other structures. This platform does not have shrinkage or compliance issues as does PPM nor does it exhibit poor host integration when compared to ePTFE but is similar to PPM in the peritoneal cavity, causing dense adhesion formation with an added complication of infection especially prevalent in multifilamented constructs. The major factors leading to hernia recurrence include insufficient mesh size to cover hernia defects, mesh disruption or extrusion caused by inadequate fixation and hematoma.

Meshes can be supplied in circular, oval, elliptical, and rectangular sheets, available in a range of sizes that can be used in their entirety or cut to size as required. Pre-cut shapes, such as Y-shaped mesh for POP repairs, or designs with openings to accommodate specific anatomical features, such as the spermatic cord in hernia repairs, are also available, saving time, reducing waste, and ensuring that the edges of the prosthesis are properly sealed. Pleated or cone-shaped mesh plugs and three-dimensional anatomically curved shapes are generally available but only suitable for use in open procedures as they cannot be compressed sufficiently to fit through narrow laparoscopic entry points.

While mesh used for laparoscopic repair is very similar to mesh that is used in open repair, there are some design differences. Laparoscopic mesh must be easy to insert through a trocar. It must also be easy to manipulate and fixate inside the human body. One limitation of biological mesh in laparoscopic hernia repair is that the thickness required for a lasting repair makes fixation very difficult with usual laparoscopic fixation devices

Polypropylene mesh, comprising a network of monofilament fibres with large pores in-between, is one of the most widely used materials. It is easily incorporated into the surrounding tissue, hence is best suited for use in areas where it will not come into contact with the abdominal viscera, as it may otherwise form dense adhesions that are difficult to remove. Although it is more inert and resistant to shrinkage than other materials, Polypropylene can undergo oxidation within the body, leading to loss of strength over time. Polypropylene meshes are also available with various coatings including titanium, which may offer improved biocompatibility, and absorbable hydrogel, used to minimise adhesions.

Polyester mesh displays greater shrinkage than other types but incorporates well and is available with a range of absorbable collagen-based coatings that can protect bowel tissue from adhesions, dissolving within around 10 to 15 days as the polyester component is incorporated.

ePTFE (expanded polytetrafluoroethylene) is a soft, flexible microporous mesh first introduced in 1970. The small pore size of less than 10 micrometres prevents cellular and fibrous ingrowth, such that integration is poor when compared to other materials, but adhesions are also less common. The material is relatively opaque, making it difficult to visualise structures on the other side of the mesh during surgery, although versions are available with larger pores for improved visualisation. Large and small pore versions can also be combined to form a double-layer material that promotes tissue growth on one side and limits adhesions on the other.

To date the ideal repair strategy remains elusive and the benefits of one mesh type versus another remain controversial. The quest for improved surgical techniques and new materials continue to be the subject of a large volume of medical literature. New constructs include an over-coating of polypropylene mesh with polyglactin produces a strong tissue reaction favoring the formation of connective tissue around the entire mesh pledget and hindering mesh incorporation. Biomaterials such as fluoropassivated gelatin-impregnated polyester mesh has been studied in vitro as a means to improve repair strength and may be beneficial in accelerating the healing process. More recently a new composite (Parietex™, Sofradim Corp.) has been introduced in the US market. The mesh is a composite of a woven Dacron polyester coated on one side with a mixture of collagen, polyethylene glycol (PEG) and glycerol that is designed to be biodegradable 3 weeks post implantation intraperitoneally. The mesh has been experimentally and clinically proven to promote quick and complete integration into tissue while inhibiting adhesion formation and visceral erosion on the abdominal side during the reperitonization period.

Mesh fixation is important for many successful open and laparoscopic repairs. The conventional methods are staples or sutures. There has been significant effort to develop optimal fasteners to improve stability and reinforcement strength]. Mechanical mesh fixation often causes tissue ischemia and possibly nerve entrapment resulting in severe postoperative pain. The use of endo-stapling devices not only increases the time to complete the surgery but can also significantly increase total cost of the procedure. The benefit of using a helical fastener for mesh fixation in laparoscopic herniorrhapy has been reported. Using cadaveric tissue, greater mesh stability with a 40% reduction in incision size was achieved. There is renewed interest in using resorbable sutures or polylactic clips (Pariefix™, Sofradim). Preliminary studies suggest a significant reduction in postoperative pain. Studies describing the use of surgical glues to either augment or replace conventional methods for mesh fixation have been reported. Both fibrin and octylcyanoacrylate were evaluated for initial bond strength and postoperative host response to the adhesive. It was found that fibrin may be as strong as staples but seems to trigger a strong fibrous reaction and inflammatory response. Similar results were observed with cyanoacrylate. There is some indication that collagen/glycosaminoglycan matrices incorporated into PPM may reduce the number of adhesions. A collagen patch coated with fibrin glue seemed to reduce time for hemostasis in the treatment of suture hole bleeding during vascular reconstruction using PTFE prostheses. Exposure of Mersilene™ sutures to a CO₂ laser improved knot strength and stability.

Postoperative Adhesions—

The formation of postoperative adhesions is widespread occurring in 55%-100% of the patient population. The most common cause results from the normal healing process at the surgical site In fact the processes that induce a strong inflammatory response and consequent tissue damage also seem to contribute to tissue repair. It is probably the imbalance between damage and repair that lead to peritoneal adhesion formation. For most patients adhesions frequently develop during the first three (3)-five (5) days following surgery. Major health problems arise from adhesion formation including intestinal obstruction, infertility and chronic pelvic pain. Several strategies to reduce the risk of adhesions have been investigated over the years including pharmacological agents and physical barriers. The effectiveness of adhesion barriers following inguinal hernia repair using coated polypropylene mesh has been reported. While there was a measurable reduction in intraabdominal adhesions, those resulting from incisional repair and peritoneal closure remained unaffected. Resorbable collagen gel and collagen/cellulose films were compared to fibrin sealant as effective barriers to postoperative adhesion formation. The materials were placed between an abdominal wall wound and a similarly sized cecal wound. At seven days, postsurgery evaluation indicated reduction in formed adhesions for both collagen and cellulose composite. However, the

most effective method for adhesion prevention is yet to be discovered. Improving microsurgical techniques to minimize tissue trauma and control of bleeding should help. Development of new biomaterials may prevent the formation of fibrin bridges further inhibiting adhesion formation.

Laser Welding Background—

There has been heightened interest in developing tissue solders and sealants as replacement for conventional closure methods, the fixation of grafts and implants and anastomoses. The advantages include speed of closure, reduced infection due to the elimination of foreign matter, acceleration of wound healing and the ease of use in laparoscopic surgery, especially when water tightness, limited access or small size of repair are important factors. Fibrin-based or albumin, crosslinked with glutaraldehyde, biomaterials exhibit low strengths and are typically used as surgical sealants. Cyanoacrylates have been used since 1960 as strong adhesives primarily for topical indications since they decompose in physiologic environments and may be toxic.

Numerous studies have reported the efficacy of light activated solders to weld soft tissues. Laser-activation provides additional benefits including a directed energy source for precise placement of the weld and is compatible with minimally invasive surgery (MIS). The availability of a variety of laser output powers, wavelengths, which match the optical properties of tissue, as well as the development of protein composites, layered solders and those modified with growth factors, chromophores, photochemicals or polyethylene glycol (PEG), have advanced the technology. Applications range from urologic anastomoses, small diameter vascular anastomoses, nerve anastomosis skin closures, liver repair and biliary reconstruction. The strength of the repair is dependent upon reaching a precise temperature set by the choice of laser and solder composition to obtain protein reconstruction at the solder/tissue interface with minimal damage to peripheral tissue. These solders tend to undergo blood dilution during surgery with mechanical alteration which weakens the repair. The stronger adhesives are brittle, inflexible and not easily adapted to different tissue geometries. Bowel closures using a Nd:Yag laser alone or with a semi-solid albumin solder are known, and the effects produced by an argon as compared to a Ho:Yag laser in intestinal anastomosis have been evaluated. While the initial strength of the laser repair appears weaker than sutures, the strengths appear identical or higher for the laser group after 7-15 days. In the laser alone repairs there was no evidence of foreign body response, less fibrosis and the presence of fewer and milder adhesions.

Often, surgical meshes are sold in sheets that have standard sizes. Prior to suturing the surgical mesh to a wound site, the clinician typically cuts the surgical mesh to a desired size and shape. For relatively large wound sites, two or more sheets are sutured and placed over the desired tissue area.

Preferably, minimally invasive procedures should be employed, using, for example a trocar, whereby the surgical mesh is inserted through a tube of the trocar and into the body cavity where it may then be sutured to the underlying tissue. For the large, irregular wound sites, multiple surgical mesh sheets, cut to a desired shape, may be required to be stitched or sutured together, as well as to the underlying tissue. Such suturing is often done inside a body cavity so as to be non-invasive. However, suturing under these circumstances can be complicated and time consuming. Moreover, the resulting stitch between meshes may be loose and weak.

### BRIEF DESCRIPTION OF DRAWINGS

FIG. 1 illustrates an example of synthetic or biologic surgical mesh that is comprised of two (2) parts: A collapsible self-forming conical shape mesh and a flat sheet of mesh designed for inguinal hernia repair;

FIG. 2 illustrates an example of the combination of the two part mesh shown in FIG. 1 by laser welding consistent with an aspect of the present disclosure;

FIG. 3 illustrates a preshaped mesh by the laser welding of a two (2) component mesh for ventral or open incisional hernia repair consistent with an additional aspect of the present disclosure;

FIG. 4 illustrates an example of a synthetic or biologic surgical mesh that is uniquely designed to include at least three (3) mesh segments that are joined together by laser welding to optimize broader coverage of the abdominal wall in ventral hernia repair consistent with an additional aspect of the present disclosure;

FIG. 5 illustrates an example of a synthetic or biologic surgical mesh that is uniquely designed to stabilize and optimize points of attachment to tissue in sacral colpopexy surgery consistent with an additional aspect of the present disclosure.

FIG. 6 illustrates an example of a synthetic or biologic or surgical mesh that has four (4) circular openings at it's corners;

FIG. 7 illustrates an example of insertion of circularly molded collagen composite glue tacks or rivets into the four (4) circular openings of the surgical mesh consistent with an aspect of the present disclosure;

FIG. 8 illustrates light exposure of the inserted molded collagen composite glue tacks or rivets for attaching the surgical mesh to tissue consistent with an additional aspect of the present disclosure;

FIG. 9 illustrates exposure of the molded collagen composite tack or rivet in which the light source is enclosed within an envelope consistent with an additional aspect of the present disclosure;

FIG. 10 illustrates the formation of cuts or notches at the four (4) corners of the surgical mesh and includes inserted molded collagen composite glue tacks or rivets to anchor the surgical mesh to tissue consistent with an additional aspect of the present disclosure;

FIG. 11 illustrates an example of a synthetic or biologic surgical mesh is patterned to include multiple openings along the implant edges for receiving the molded collagen composite glue tacks or rivets for attaching and stabilizing the surgical mesh to tissue consistent with an additional aspect of the present disclosure;

FIG. 12 illustrates an example of a synthetic or biologic surgical mesh that has a pattern of multiple openings over the entire implant or surgical mesh surface for receiving the molded collagen composite glue tacks or rivets for maximizing the attachment of the implant or surgical mesh to tissue consistent with an additional aspect of the present disclosure; and

FIG. 13 illustrates an example of a synthetic or biologic surgical mesh that has openings formed by cutting slits at the edges of the implant to increase the surface area of the molded collagen composite glue tack or rivet to stabilize the attachment of the implant to tissue consistent with an additional aspect of the present disclosure.

FIGS. 14-18 illustrate steps in accordance with a method consistent with the present disclosure.

### DETAILED DESCRIPTION OF THE INVENTION

Consistent with the present disclosure a method is provided in which a plurality of surgical meshes are provided, each of which may include a coating that has a chromophore. Alternatively, each mesh has a plurality of rivets or tacks that include the chromophore. The meshes may then be positioned to overlap one another or be provided adjacent one another inside the body cavity such that the mesh cover the wound site. The meshes may then be exposed to light at a wavelength that activates the chromophore and causes the meshes to adhere to one another and the underlying tissue. In one example, the coating or the tacks includes a combination of relatively high concentration derivatized collagen and riboflavin, lumiflavin or lumichrome.

The exposure takes relatively little time and the resulting bond may be stronger than that associated with sutures. As a result, large, irregular meshes can be formed inside the body cavity from smaller meshes, but in a much faster procedure than that associated with sutures. Moreover, the procedure is less complicated since suturing is not necessary and bonding occurs simply by exposure to light. Alternatively, if desired, limited suturing may be performed, such as in a few spaced locations along the periphery of each mesh. In either case, the required to attached the surgical meshes may be significantly reduced, thereby minimizing cost and increasing the likelihood of a positive outcome for the patient.

Reference will now be made in detail to exemplary embodiments of the disclosure, examples of which are illustrated in the accompanying drawings. Wherever possible, the same reference numbers will be used throughout the drawings to refer to the same or like parts.

Collagen that incorporates a photosensitive chromophore riboflavin, for example, that functions as a thin coating is incorporated directly to a synthetic surgical mesh, as described, for example, in U.S. Patent Application Publication No. 2011/0125187, the entire contents of which are incorporated herein by reference. It is believed that riboflavin is released to the tissue surface, and, during such release, the collagen layer is exposed to light (e.g., light having a wavelength between 365-375 nm or 440-480 nm), resulting in attachment to the tissue surface. In other examples, the collagen may instead contain lumichrome or luminflavin.

Consistent with an aspect of the present invention, a method of attaching a surgical mesh to repair defective tissue or use in reconstructive surgery using a photoactivated collagen-based tissue adhesive is disclosed. Examples of a surgical mesh includes polypropylene mesh, comprising a network of monofilament fibres with large pores in-between, is one of the most widely used materials. Another example is a polyester mesh which exhibits greater shrinkage than other types but incorporates well. Another commonly used mesh construct, Eptfe (expanded polytetrafluoroethylene), is soft, flexible and microporous. The small pore size of less than 10 micrometres prevents cellular and fibrous ingrowth, such that integration is poor when compared to other materials, but adhesions are also less common.

Consistent with a further aspect of the present disclosure is a collagen coating which is comprised of a composition that includes a collagen solution with added photosensitive chromophores such as riboflavin. Once the chromophore is dissolved within the collagen solution, the solution is poured into a mold of different sizes or shapes and once gelatinized demolded and ready for integration with the opaque implant or surgical mesh substrate. Alternatively, the chromaphore may include lumiflavin or lumichrome. This invention also includes exposing the layer to optical energy having a wavelength in a range of 365-375 nm or 440-480 nm.

Consistent with an additional aspect of the present disclosure, a collagen composite may be applied to attach a surgical mesh to tissue. The collagen composite may include collagen that includes riboflavin. The composition may be molded into a desired shape that can be incorporated within a biologic, or synthetic surgical mesh and applied as a layer to a surface of tissue that is in need of repair. Alternatively, the composition of collagen may include luminflavin or lumichrome. The invention may also include directing a beam of radiation toward the collagen composition that includes riboflavin incorporated within the surgical mesh and provided to the tissue surface such that the beam exposes the composition to the radiation, the radiation including light having a wavelength in a range of 440-480 nm.

Consistent with a further aspect of the present disclosure, a method is disclosed in which a collagen composite cylindrical tack or rivet is incorporated into a synthetic or biologic surgical mesh. The method comprises a step of incorporating the surgical mesh with a composition of collagen within the surgical mesh, the collagen composition including lumiflavin, which is exposed to optical energy having a first wavelength in a range of 440-480 nm or in a range of 300 nm-410 nm to tack-weld the surgical mesh to repair or reconstruct tissue defects. The method may further includes a step of directing a beam of radiation toward the composition provided on the surface of the surgical mesh such that the beam exposes the collagen composition in the form of a cylindrical tack or rivet to the radiation, the radiation including light having a wavelength in a range of 440-480 nm.

Consistent with the present disclosure, several benefits as compared to the methods currently used for the fixation of synthetic or a biologic surgical mesh to tissue which includes sutures or staples may be realized. The collagen incorporated chromophore can conform to any shape or thickness and conforms to curved, flat or irregular tissue surfaces. The collagen compositions can be varied as well as the concentration of the chromophore to optimize the delivery of the chromophore to the target site. An optimal dose of the chromophore may be delivered to the attachment site, so that once activated with light, it causes strong attachment of the implant or surgical mesh to the tissue site. The solubility of the chromophore is enhanced in the process of incorporation within the collagen composition. Other advantages include preventing the chromophore from migrating or diffusing to adjacent healthy tissue so as not to dilute the chromophore concentration at the attachment sight. The blue light is so contrived that it only impinges on the solder composite tacks or rivets. The light penetration is controlled to activate only the released chromophore at the site.

The accompanying drawings, which are incorporated in and constitute a part of this specification, illustrate several embodiments of the present disclosure and together with the description, serve to explain the principles of the disclosure.

A molded shape of collagen is disclosed that incorporates a photosensitive chromophore riboflavin, for example, that functions as a tack or rivet is incorporated directly to a synthetic or biologic surgical mesh While the riboflavin is released to the attachment tissue surface, the collagen layer is exposed to light (e.g., light having a wavelength between 365-375 nm or 440-480 nm). In other examples, the collagen may instead contain lumichrome or luminflavin.

The collagen composite coating is preferably made from a collagen which has been extracted, purified, solubilized, chemically modified and reconstituted in accordance with techniques described in U.S. Pat. Nos. 6,773,699 and 6,875,427, the entire contents of both of which are incorporated herein by reference. Preferably, the starting collagen is prepared from porcine corium, however other sources of collagen may also be used. Porcine hide is rinsed with reagent alcohol to reduce bioburden. The hide is cut into sections approximately 24 inches wide and passed through a “splitter” to remove epidermis and underlying membranous tissue. Split hide is rinsed with reagent alcohol and placed in frozen storage prior to processing. Sections of split corium are cut into small pieces (about 1 cm²) and soaked in reagent alcohol and then washed extensively with sterile water. The washed pieces are placed in 20 volumes of 0.5M HCl for 30 minutes, washed with sterile water and then placed in 20 volumes of 0.5N NaOH for 30 minutes. Both treatments have been shown to be effective in reducing viral titers by up to 6 logs. In addition, both treatments have been shown to have significant bactericidal effects, reducing bacterial loads by up to 9 logs. The chemically disinfected corium is washed extensively in sterile water, weighed and placed in 20 volumes (v/w) of 0.5M acetic acid. The pieces are stirred for 72 hours and porcine mucosal pepsin added to the partially swollen corium.

Pepsin is added at 2% (w/w wet corium) and stirred for 48 hours. An additional aliquot of pepsin is added at 1% (w/w wet corium) and stirred for another 24 hours. At this point, the corium is “dissolved” in acetic acid. Small, undissolved pieces are removed by filtering the thick slurry through cheesecloth. The filtrate is diluted with 0.5M acetic acid and dialyzed against 0.5N acetic acid using dialysis tubing having a 50,000 dalton nominal cut-off. This process removes pepsin and degraded pepsin. The retained liquid containing collagen is subjected to differential NaCl precipitation to isolate predominantly Type I collagen. Purified Type I collagen at about 5 mg/MI is then dialyzed against 0.1N acetic acid. The retained collagen solution is subsequently filtered through 0.45 μm and 0.2 μm filters. Twenty-four (24) liters of collagen stock solution are stored at refrigeration temperatures. Total collagen yield is approximately 120 grams.

Hydroxyproline analysis has determined the pure collagen concentration is 4.88 mg/MI and the UV absorbance profile at 280 nm indicated no presence of pepsin (sensitivity 1 part per billion). SDS-PAGE and Differential Scanning calorimetry (DSC) is conducted to examine the purity of Type I collagen. DCS profiles show transition temperatures of 43° C. indicative of undenatured molecular collagen.

Purified, telopeptide-poor Type I collagen is derivatized with glutaric anhydride. The anhydride reacts with deprotonated free amines and substitutes a carboxyl group for the reacted amine group, making the composition anionic. The degree of derivatization is selected so that the modified collagen remains soluble at physiologic Ph. Derivatization is performed by adjusting the Ph of soluble collagen (5 mg/MI) to 9.0, using NaOH, adding solid anhydride to the collagen at different concentrations ranging from 10%-30% (w/w) solution while maintaining the Ph at 9.0 during the reaction. After 15 minutes, the Ph of the solution is reduced to about 4.5 to precipitate derivatized collagen. The precipitate is recovered by centrifugation at 14,500 RPM for 20 minutes and 9° C. The precipitate is washed two times with sterile water. The final precipitate is dissolved in 5 Mm phosphate buffer at Ph 7.2 at a final concentration of 5 mg/MI. The solution is freeze dried in trays at a controlled rate.

Collagen coatings and films are prepared from the lyophilized derivatized collagen. Lyophilized sheets are cut into small pieces and homogenized in a Tekmar Tissue mill. Gelatinized layers are prepared by dissolving collagen powder in sterile water phosphate buffer at Ph 7.2. The collagen solid concentration ranged from 10%-60% and were obtained by exposing the dispersions to a controlled temperature water bath. As the collagen dissolved, more powder is added until the desired concentration is achieved (weight to volume). At this step the chromophore is added as a solid powder to a desired concentration (weight to volume) with continued stirring in a controlled temperature water bath. Once the desired concentration is achieved, the collagen solution is centrifuged and applied to one surface of the surgical mesh. While still warm a Teflon plate may be pressed onto the surface coated surgical mesh to control the thickness of the coating on the mesh. The coating is pressed through the surgical mesh pores for uniform coating of the mesh underside. Coating thicknesses may range from 100 μm to 2 mm. After cooling for 3 min the uniformly coated surgical mesh is removed from the press, vacuum packaged, sterilized, labeled and stored at 4° C. until use.

Preferably the collagen composite coating includes a collagen concentration of 10-60% and the concentration of chromophore, eg. Riboflavin is 0.1 to 1.0 percent and preferably 0.5%. It is noted that the collagen composite coating can coat circular shapes or alternative shapes such as square shapes, rectangular shapes or triangular shapes.

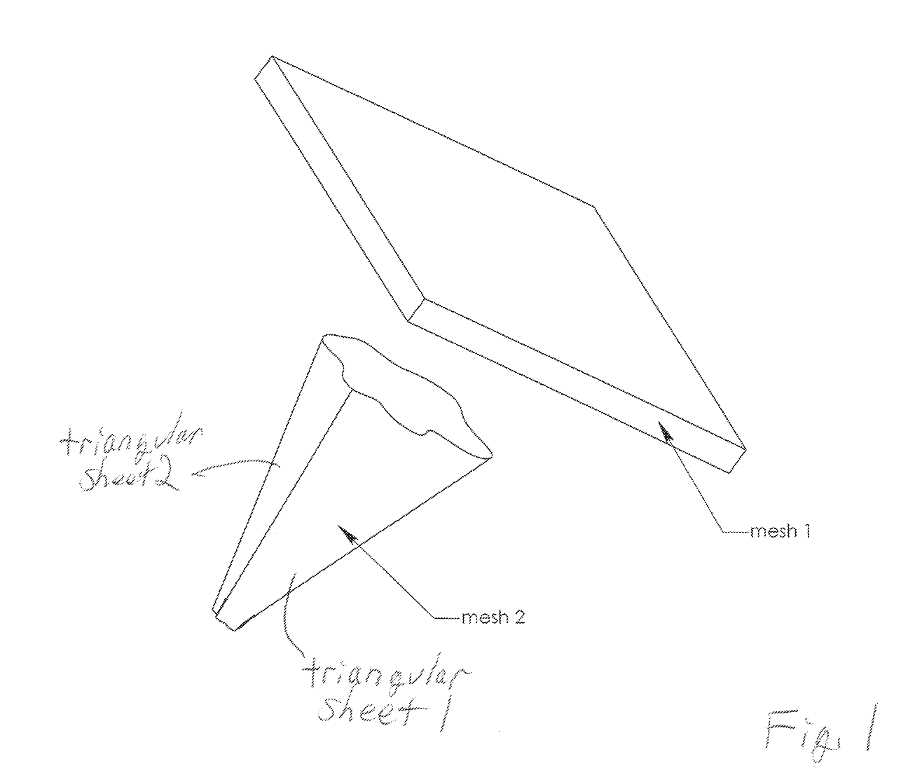

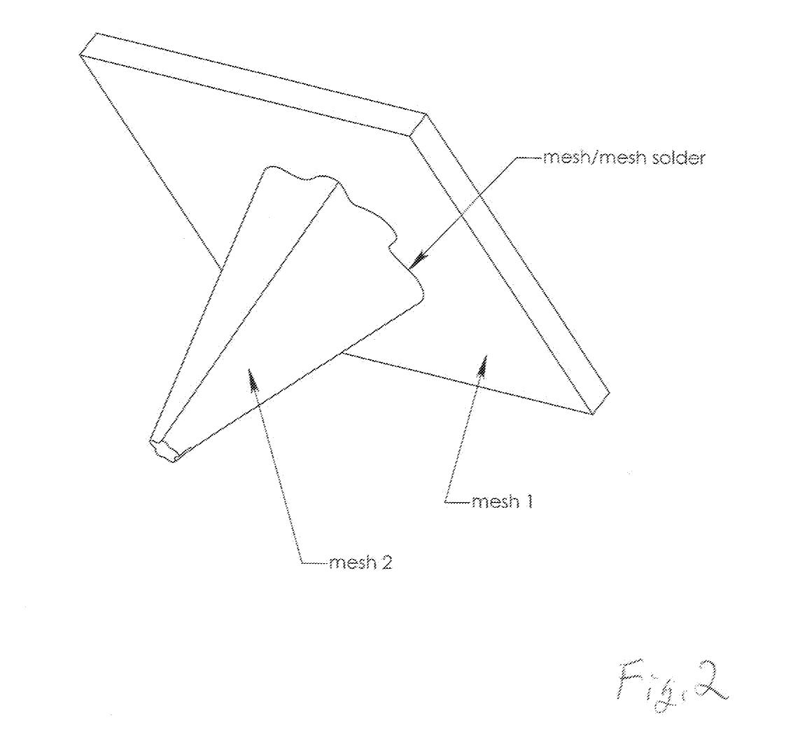

FIG. 1 shows an exemplary synthetic or biologic surgical mesh that is comprised of two (2) parts. The collagen composite coated conical shaped component, mesh 2, can be preshaped by laser welding multiple triangular sheets together to fabricate the optimal conical size as a plug to insert into a defect in a tension-free incisional inguinal repair strategy. The addition of an onlay, mesh 1, over the conical plug prevents future herniation if surrounding tissue becomes deficient. When the size of the collagen composite coated conical plug, mesh 2, is too large to fit into a standard trocar for laparoscopic repairs, collagen composite coated triangular sheets that comprise the conical plug can be individually inserted and the onlay collagen composite coated mesh 1 can be rolled, compressed and also inserted into a trocar. Once the triangular collagen composite coated triangular sheets are in the vicinity of the repair, the laser laparoscopic device can deliver optical energy to attach or glue the individual meshes together, such that the conical plug optimally fits the defect. For example, as shown in FIG. 2, the onlay collagen composite coated mesh 1 is joined to the conical shaped plug and this assembly is then inserted into the defect to complete the repair. The combined assembly comprised of collagen composite coated synthetic or biologic surgical meshes preferably conforms to the surface of the tissue where a lesion or defect may be located.

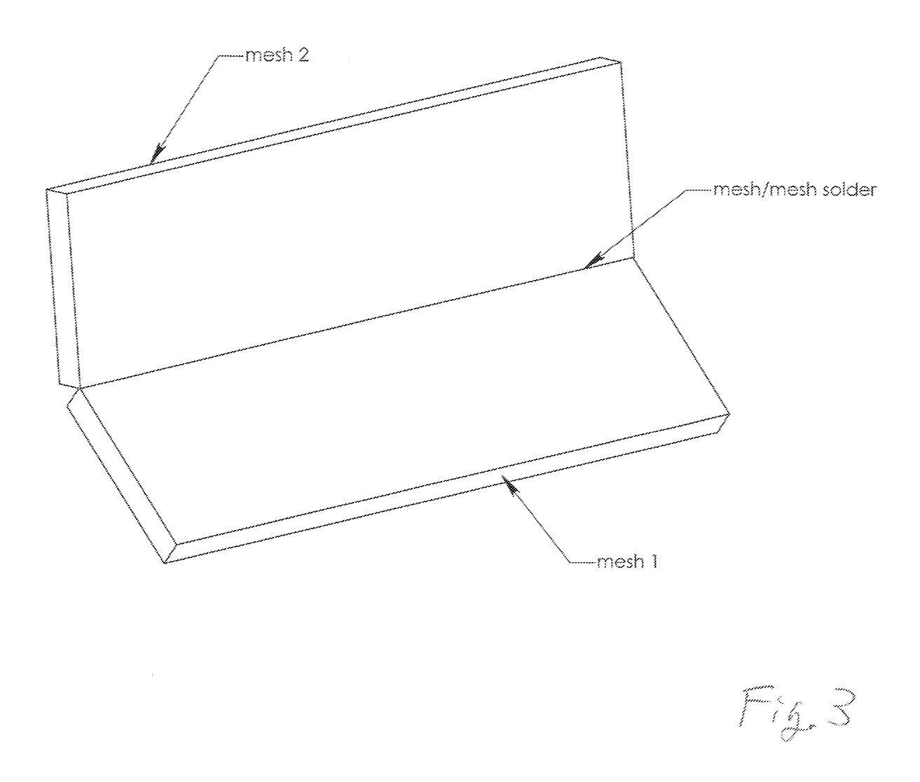

FIG. 3 shows an example in which a collagen composite coated synthetic or biologic surgical mesh 1 is joined to a second collagen composite coated synthetic or biologic surgical mesh 2 by exposing the junction between the two (2) meshes to a beam of light having a wavelength between 365-375 nm or 440-480 nm. Preferably, the light source has a wavelength of 450 nm. The light source may include a laser or light-emitting diode (LED), for example. The design of this invention may assure a sufficient mesh overlap of the defect in a ventral or incisional hernia repair strategy, for example. This preshaped mesh design eliminates the complicated maneuvers associated with suturing the mesh together and followed by fixation of the device to the peritoneal wall by additional complicated suturing.

For laparoscopic ventral hernia repair strategies the two meshes may be inserted in a trocar separately especially when the size of the meshes are larger that the trocar opening even when the meshes are compressed. The two meshes may then be joined together as shown in FIG. 3 at the site of the defect by delivering the light energy laparoscopically and then applying to the peritoneal wall to assure coverage and overlap of the defect site. This assembly is then fixed to the peritoneal wall by exposing the entire two-part device to light energy in the wavelength range between 365-375 nm or 440-480 nm. Preferably, the light source has a wavelength of 450 nm. In the example shown in FIG. 3, broader coverage of the abdominal wall may be obtained which may result in tension free repair.

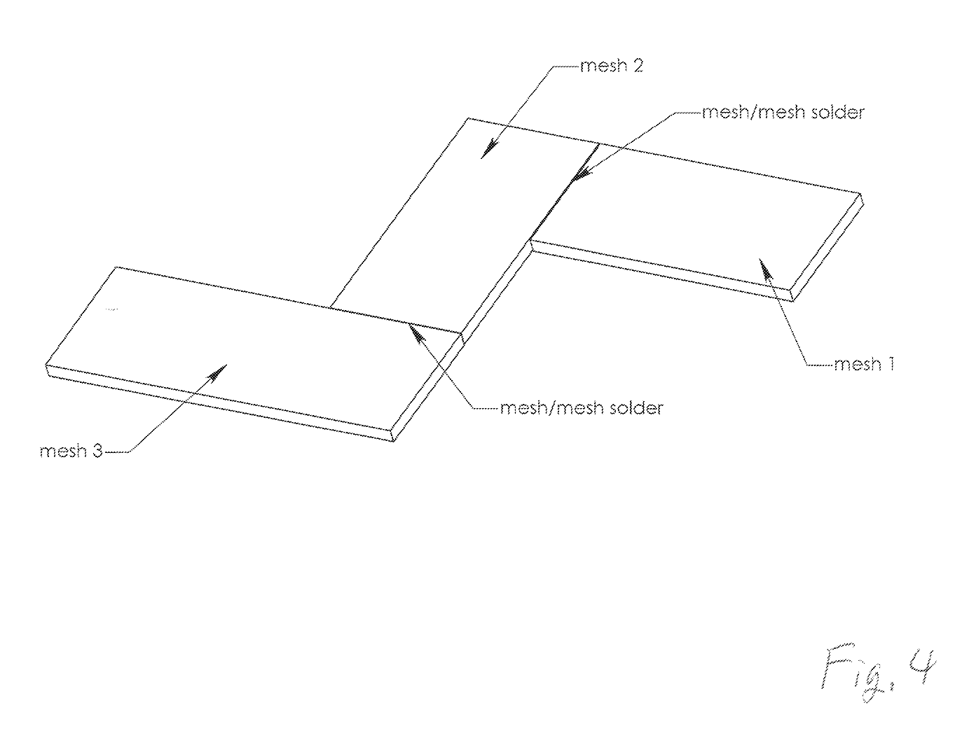

FIG. 4 shows another example in which three collagen composite coated mesh segments are joined by laser welding to assure adequate mesh overlap for a complex shaped defect in a ventral or incisional hernia repair strategy. For laparoscopic complex ventral hernia repair strategies the three meshes may be inserted in a trocar separately especially when the size of the meshes are larger that the trocar opening even when the meshes are compressed. The three meshes are then joined together as shown in FIG. 4 at the site of the defect by delivering the light energy laparoscopically, for example, and then applying to the peritoneal wall to assure coverage and overlap of the defect site. The assembly is then anchored to the peritoneal wall by exposing the entire three-part device to light energy in the wavelength range between 365-375 nm or 440-480 nm. Preferably, the light source has a wavelength of 450 nm.

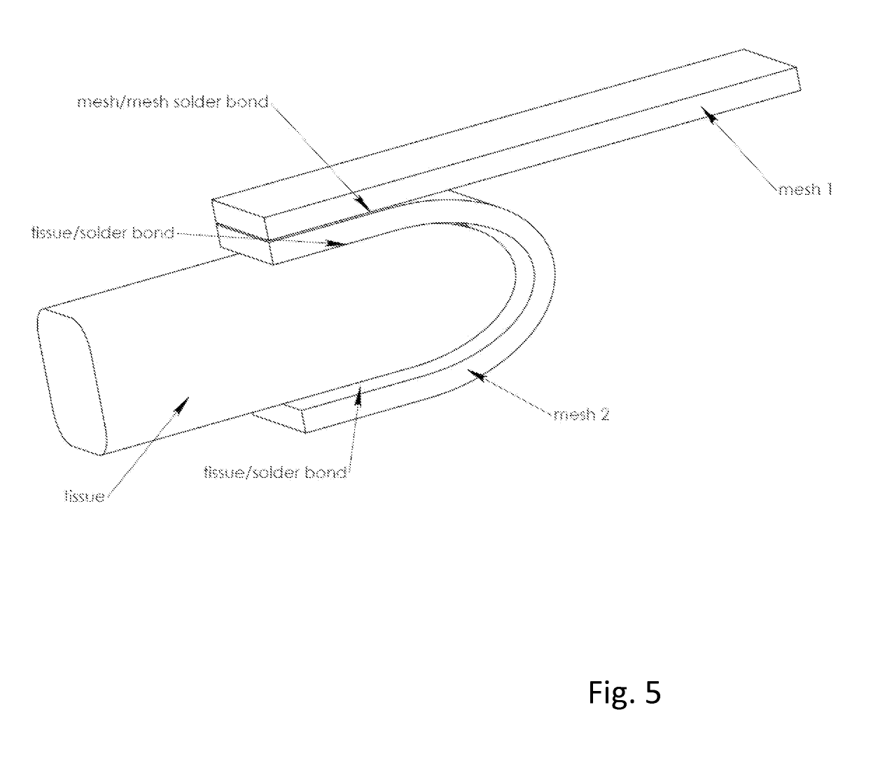

FIG. 5 illustrates further example in which two collagen composite coated mesh segments, for example, are joined by laser welding to obtain a three point fixation in a sacral colpopexy strategy. Surgical treatment involves the bonding of the collagen composite coated surgical mesh 2 segment at two fixation points, for example, on opposite sides of the vagina. The vagina may then stabilized by anchoring the intervening collagen composite coated mesh 1 to the sacrum. Mesh 1 may be joined to the mesh 2 segment by laser welding pre-operatively in an open procedure. If the mesh size and shape prevents ease of insertion into a standard trocar for laparoscopic sacral colpopexy, the two mesh segments can be joined together at the repair site using laser welding. Light energy in the wavelength range between 365-375 nm or 440-480 nm and preferably a wavelength of 450 nm is directed through the trocar to expose the interface between the collagen composite coated mesh 1 and collagen composite coated mesh 2 interface to combine the two (2) mesh segments together. The mesh configuration may then glued to tissue using the same light source.

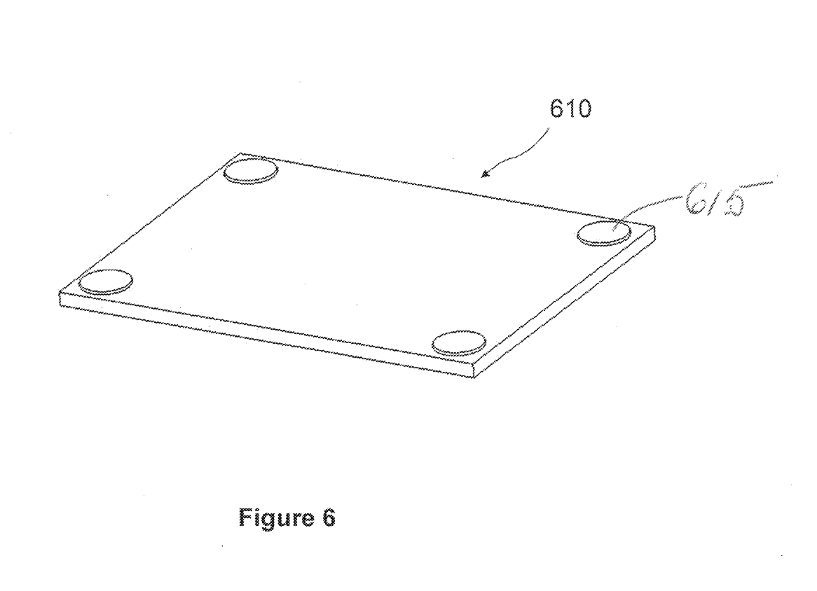

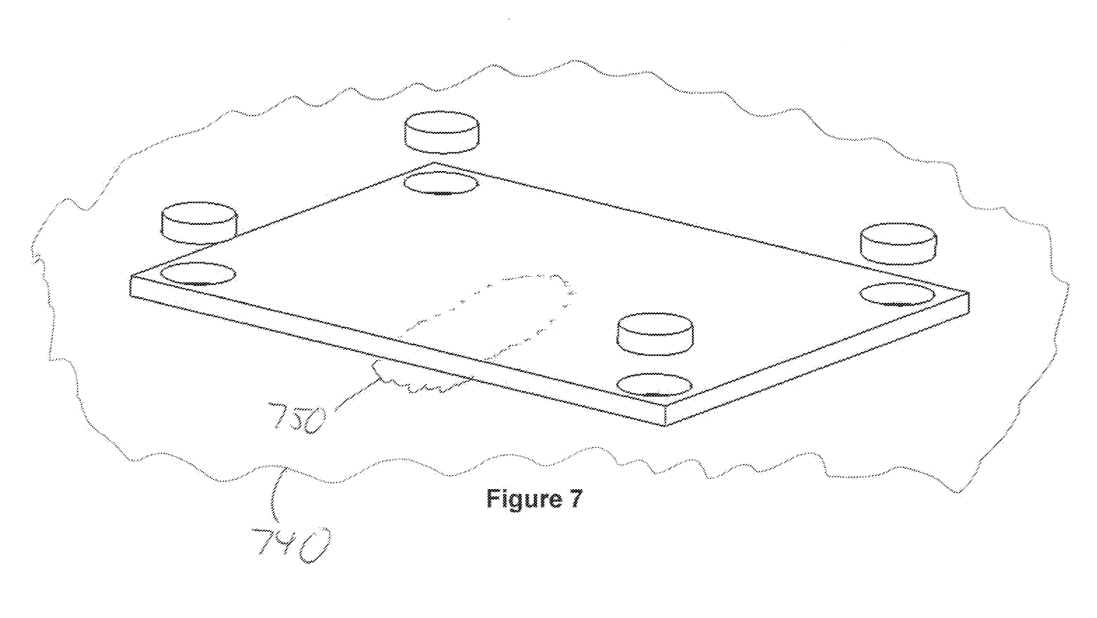

FIG. 6 shows an exemplary synthetic or biologic optically opaque implant or surgical mesh 610 with circular openings 615 created at the corners of the implant or surgical mesh. As further shown in FIG. 7 the circularly molded collagen composite tack or rivet 720 may be inserted into the circular openings 615 of the synthetic or biologic optically opaque implant or surgical mesh 610 which is then placed on tissue 740 having a lesion or defect 750. The human tissue may constitute part of the abdomen such as for hernia repair. The combined synthetic or biologic optically opaque implant or surgical mesh 610 preferably conforms to the surface of the tissue 740 where lesion or defect 750 is located

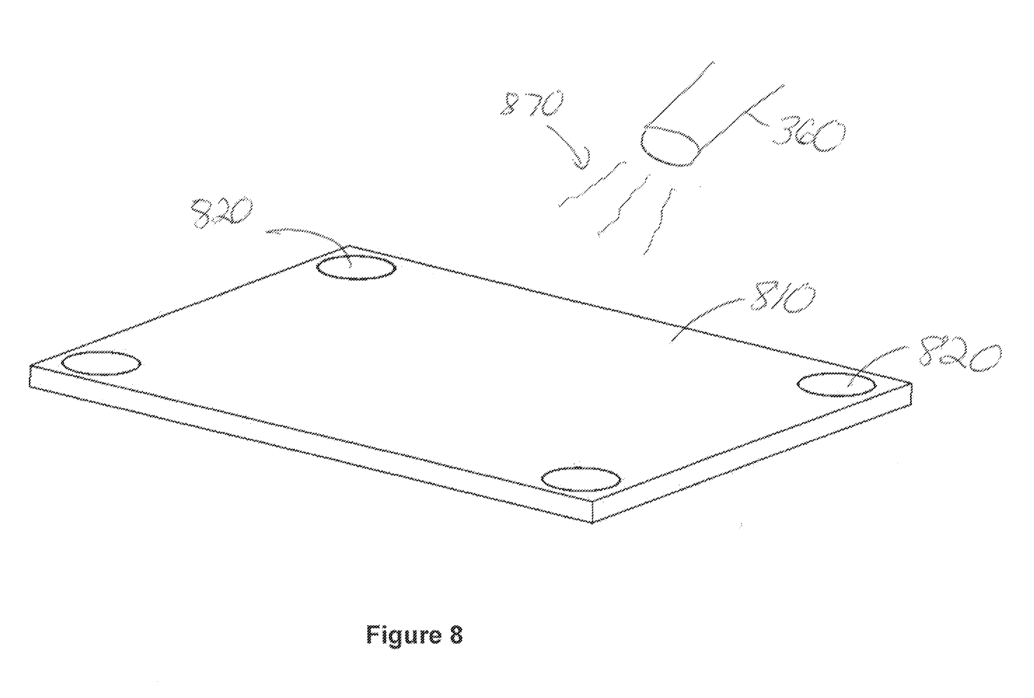

FIG. 8 shows a first example in which a synthetic or biologic optically opaque implant or surgical mesh 810 is combined with collagen composite tacks or rivets 20 and exposed to light source 860. Here, source 360 is spaced from and directs optical energy or light 870, typically in a range of 440 to 480 nm towards collagen composite tack or rivet 820. Preferably, light 370 has a wavelength of 450 nm and source 360 includes a laser or light-emitting diode (LED), for example.

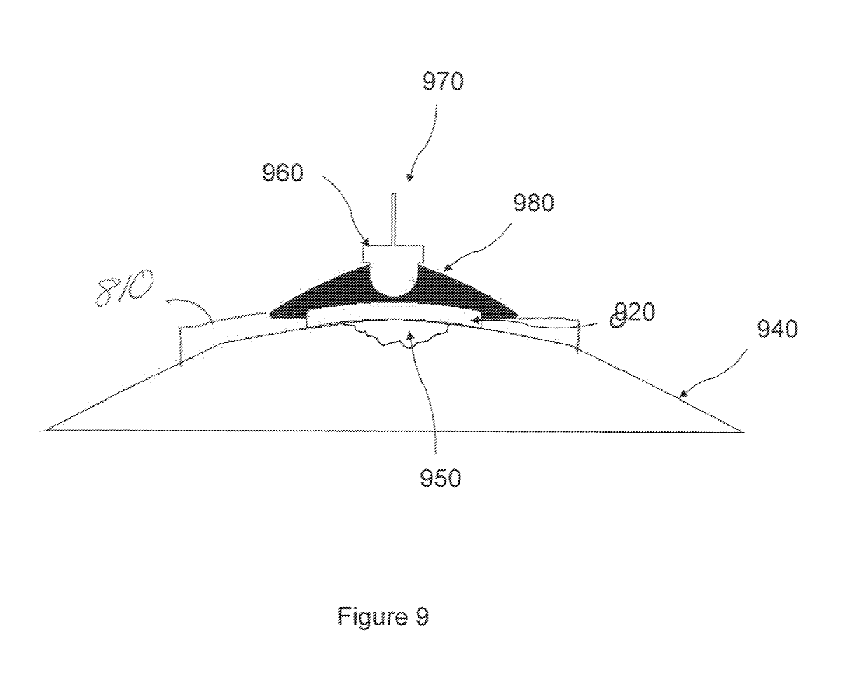

FIG. 9 shows a second example in which source 970 includes an LED 960 or laser provided within an envelope 980. Preferably, envelope 980 is provided on tissue 940 such that envelope 980 covers collagen composite tack or rivet 820 and LED 960 is positioned over lesion or defect 950. Typically, LED (or laser) 960 is centered over collagen composite tack or rivet 920. In this example, source 960 may be brought within relatively close proximity to collagen composite tack or rivet 920, so that light may be accurately directed toward the collagen composite tack or rivet 920. A reflective coating may be optionally provided on an internal surface of envelope 980 so that light is efficiently supplied to collagen composite tack or rivet 920 and is not absorbed by envelope 980. In addition, envelope 980 may have similar dimensions as collagen composite tack or rivet 920 so that light is not supplied to portions of tissue 940 unaffected by the lesion or defect 950.

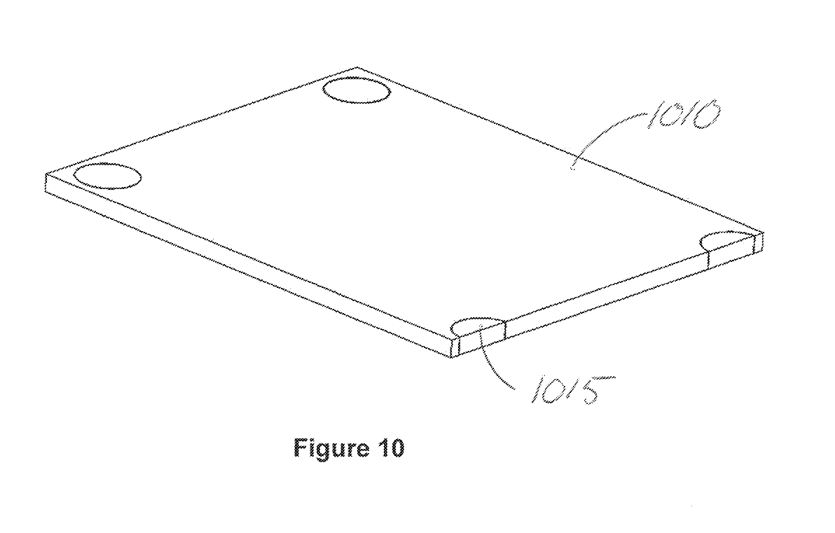

FIG. 10 illustrates another example of a circular opening 1015 notched or cut at the edges of the four corners of the synthetic or biologic optically opaque implant or surgical mesh 1010 so that the circularly molded collagen composite tack or rivet can easily be inserted from the side of the synthetic or biologic optically opaque implant or surgical mesh 1010 as compared to insertion of the molded collagen composite tack or rivet from the top of the implant 1010 in order to securely anchor the implant to tissue 1040.

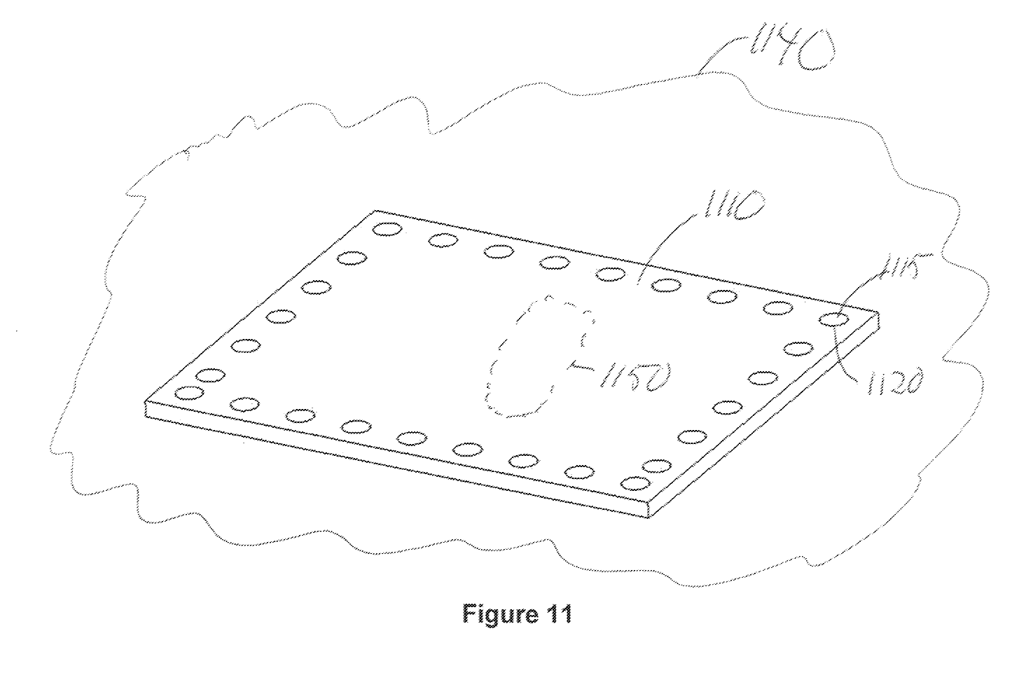

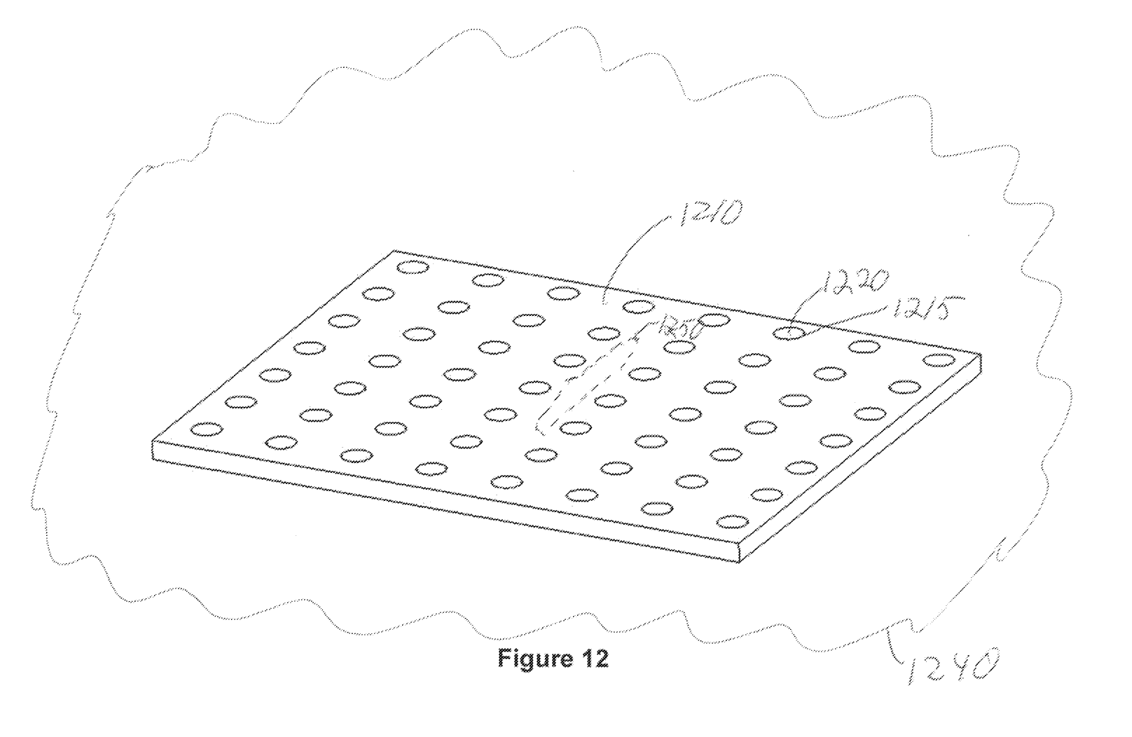

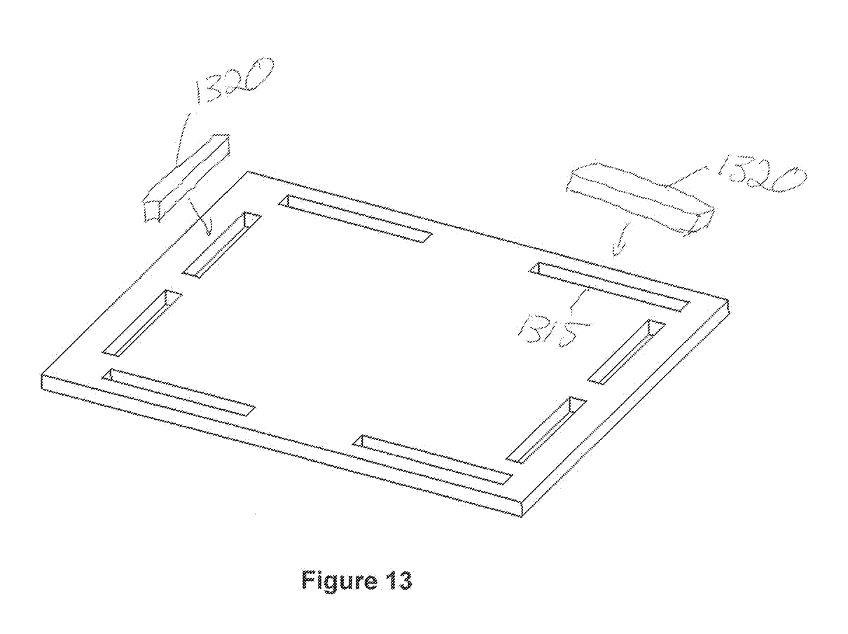

Alternatively, as shown in FIG. 11, multiple circular openings 1115 are created along the four (4) edges of the synthetic or biologic optically opaque implant or surgical mesh 1110 as receptacles for multiple circularly molded collagen composite tacks or rivets 1120 in order to maximize strength of attachment of the synthetic or biologic optically opaque implant or surgical mesh 1110 to the underlying tissue 1140 to repair a lesion or defect 1150. In FIG. 12, multiple circular openings 1215 are shown to cover the entire synthetic or biologic optically opaque implant or surgical mesh 1210 in order to accept multiple circularly molded collagen composite tacks or rivets 1220 for multiple points of attachment to the underlying tissue 1240 that has a single or multiple lesions or defects 1250. FIG. 13 shows an example in which rectangular shaped slits 1315 are cut at all four edges of the synthetic or biologic optically opaque implant or surgical mesh 1310 to receive four rectangular shaped collagen composite tacks or rivets 1320, two of which are shown in FIG. 13, to further enhance the attachment strength of the implant or surgical mesh 1310 to underlying tissue 1340 that has a single or multiple lesions or defects 1350.

In one example, the collagen composite tacks or rivets disclosed above include a 0.5% riboflavin concentration, and the exposure time to light 460 is for approximately 5 minutes. The exposure may also be for duration of in a range of 5 minutes to one hour, and the intensity of light 460 may be within a range of 1.5-70 Mw/cm². In addition, the underlying tissue is preferably maintained in a fixed position during the exposure, and the exposing light may be in the form of collimated beam.

As discussed above, the collagen composite tack or rivet consistent with the present disclosure may include riboflavin. However, it is also contemplated that the collagen composite tack or rivet may include lumichrome or lumiflavin instead. In a further example, the collagen composite tack or rivet may be preexposed to include preactivated riboflavin, which has been exposed to ultraviolet light having a wavelength in a range of 300 nm to 410 nm or blue light having a wavelength in the range of 440-480 nm prior to application to the tissue. In addition, the collagen composite tack or rivet 720 may be applied to the tissue in a manner similar to that discussed above in reference to FIG. 6. Moreover, the same or similar exposure parameters (intensity, wavelength, and exposure duration) may also be employed and the sources discussed above in regard to FIGS. 3-4 may be used.

The collagen composite tack or rivet may adhere to the tissue, but may dissolve by fluids present in and around the tissue after a short period of time. In particular, depending on the concentration of collagen in the collagen composite tack or rivet, the amount of time required for the collagen composite tack or rivet to dissolve may vary from approximately 5 minutes to approximately 30 days. Thus, an advantage of the collagen composite tack or rivet is that it remains tacky to tissue for a time sufficient to perform the exposure discussed above, and thereafter, harmlessly dissolves once the synthetic or biologic optically opaque implant or surgical mesh is stabilized by bioincorporation. There is no need for a practitioner to remove any remaining collagen composite tack or rivet once it is applied.

It is believed that the antiseptic properties of the above-described exposed collagen composite tack or rivet incorporated with a chromophore stem from release of oxygen free radicals in combination with the generation of nucleotides that preferentially interrupt the RNA or DNA of pathogens that cause bacterial infections. Accordingly, it should also be effective in a broad spectrum of pathogens, including bacteria, viruses, parasites and fungi. Due to the mechanism of action, development of resistance is unlikely. In addition, it has been observed that the level of bacterial infection has been significantly reduced after a single treatment. This might avoid the need for antibiotics following surgical interventions.

The above described tack or rivet patterns shown in FIGS. 6-13 are exemplary. Other patterns are contemplated to accommodate various tissue shapes and contours. For example, a tissue may have a recessed portion that is difficult for a portion of the mesh to attach to. That part of the mesh may be uncoated in order to minimize cost. On the other hand, those portions of the mesh that can positioned to readily contact an underlying tissue may be coated, and those coated portions may be attached to corresponding underling tissue accordingly.

As discussed above, the collagen composite synthetic or biologic mesh coating may include riboflavin. However, it is also contemplated that collagen composite coating may include lumichrome instead. Alternatively, the collagen composite coating may include lumiflavin. In a further example, the collagen composite coating may be preexposed to include preactivated riboflavin, which has been exposed to ultraviolet light having a wavelength in a range of 300 nm to 410 nm or blue light having a wavelength in the range of 440-480 nm prior to application to tissue.

As further noted above, collagen composite synthetic or biologic mesh coating adheres to tissue, but may be dissolved by fluids present in and around the tissue after a short period of time. In particular, depending on the concentration of collagen in the collagen composite coating, the amount of time required for the collagen composite coating to dissolve may vary from approximately 5 minutes to approximately 30 days. Thus, an advantage of the collagen composite coating is that it remains tacky to tissue for a time sufficient to perform the exposure discussed above, and thereafter, harmlessly dissolves once the synthetic or biologic surgical mesh is stabilized by bioincorporation. There is no need for a practitioner to remove any remaining collagen composite coating once it is applied.

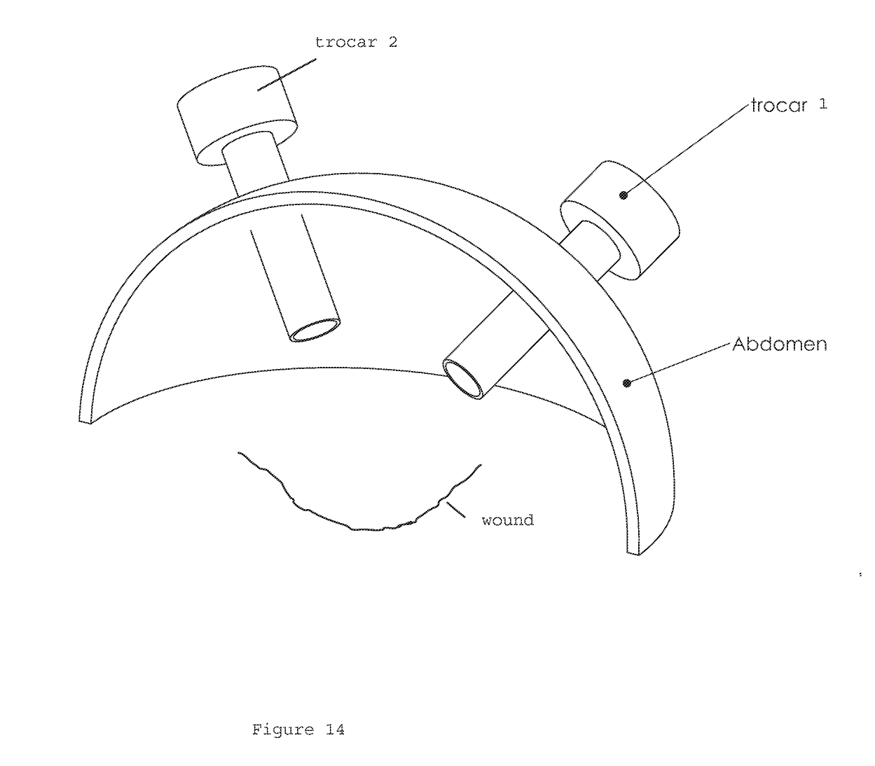

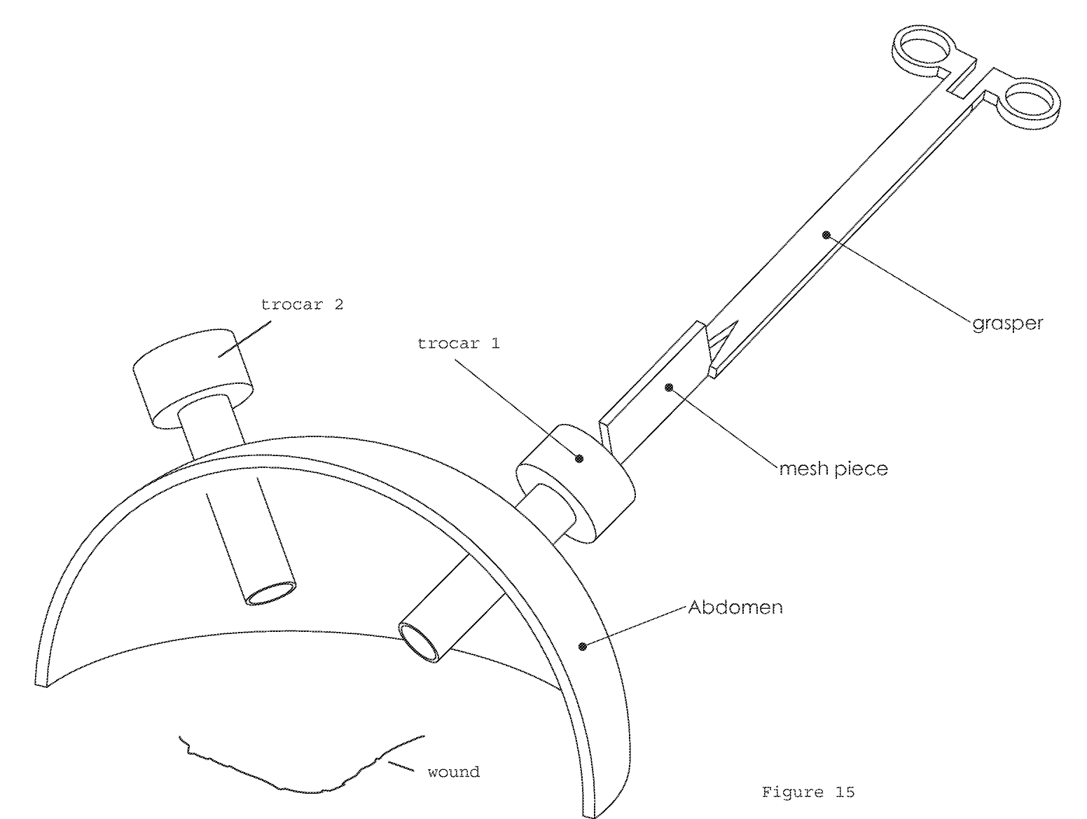

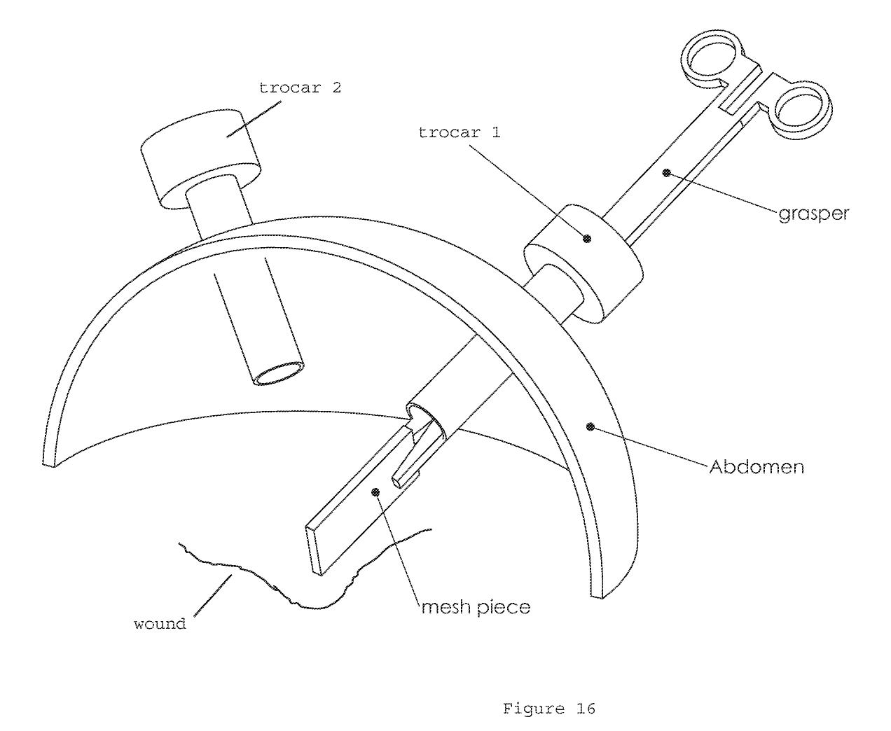

FIGS. 14-18 illustrate exemplary steps of a method consistent with the present disclosure. FIG. 14 illustrates a trocor that has been inserted into an inflated cavity. In FIG. 15, a surgical meshes is held by a grasper outside the cavity. In FIG. 16, the grasper is used to insert the mesh into the cavity through trocar 1. One or more additional meshes may be inserted into the cavity in this manner. As noted above, each of the plurality of meshes may include a coating that includes a chromophore or tacks that include the chromophore.

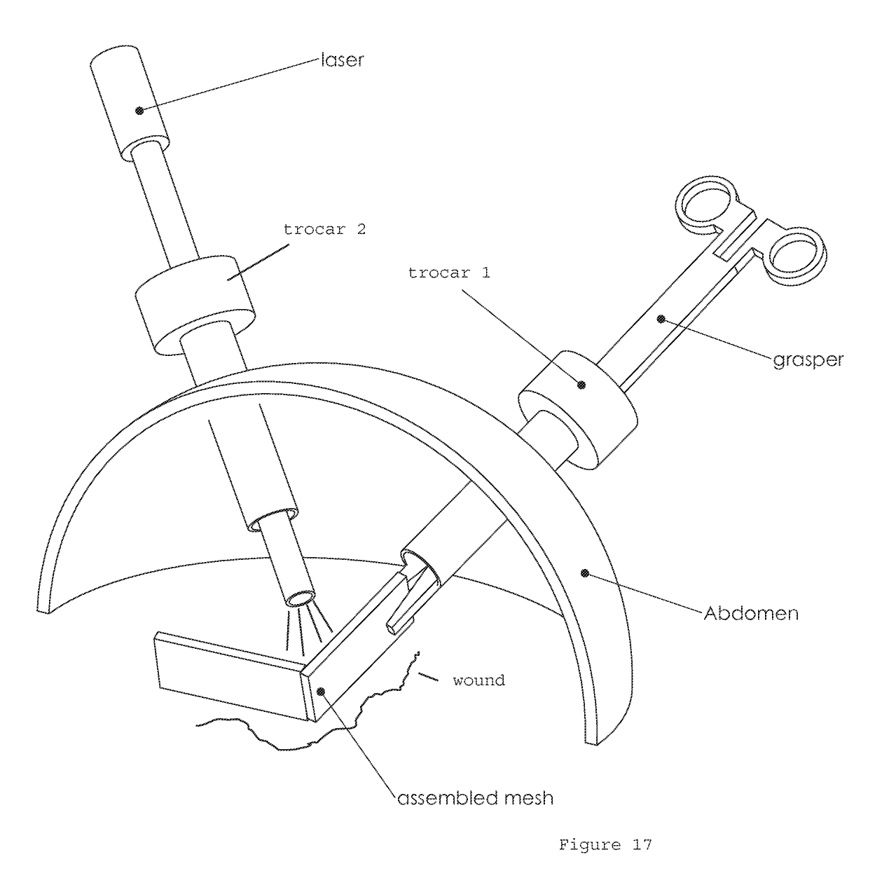

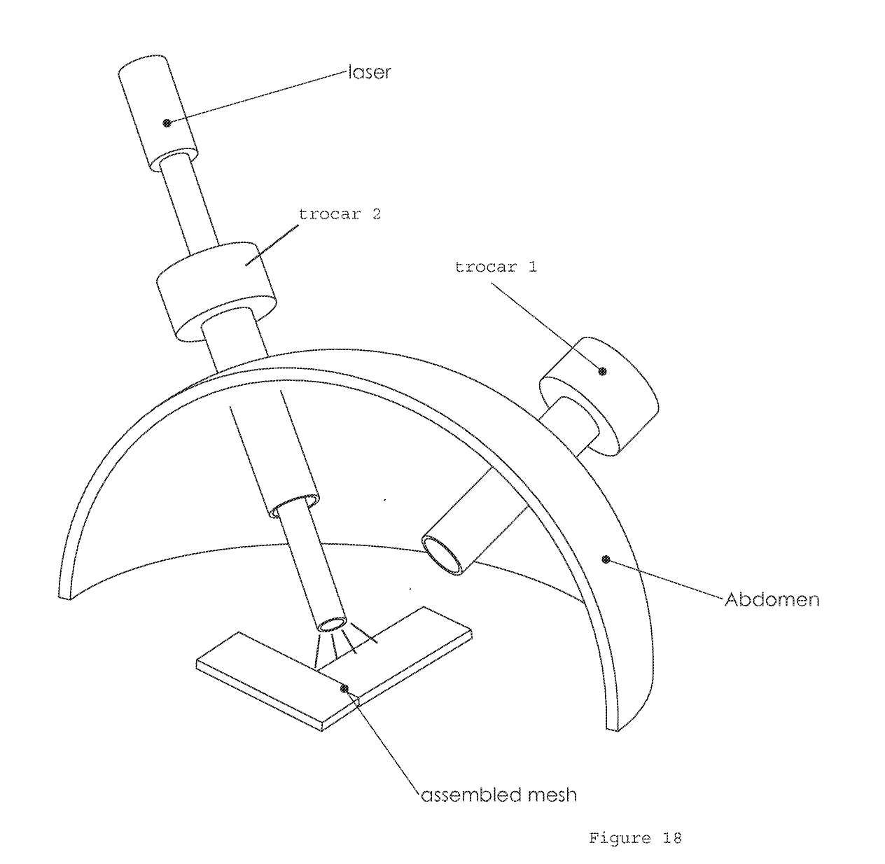

As shown in FIG. 17, first and second meshes are aligned to be adjacent one another. Alternatively, the meshes may overlap one another, such the tacks of one mesh overly and contact the tacks of the other mesh. Next, as shown in FIG. 18, the meshes are exposed to light from a laser light source provided in trocar 2, e.g., a semiconductor laser or fiber laser, to activate the chromophore, such that the meshes adhere to one another along a seem provided at the junction of the meshes and bond to the underlying tissue. In another example, the tacks are exposed to bond or attached one mesh to the other, as well as to the underlying tissue.

In another example, the meshes may be bonded, outside the cavity, to one another by gluing, e.g., exposing the meshed to light having a predetermined wavelength that activates the chromophore, as well as tacking or riveting the meshes to one another, as discussed above. In a further example, the meshes may be bonded outside the cavity. In addition, although the light source is described above as being a laser, it is understood that light source may also include a light emitting diode (LED).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method, comprising:
providing a plurality of surgical meshes, each of the plurality of surgical meshes including a material that has a chromophore;
inserting the meshes into a body cavity;
providing the plurality of meshes on a wound site in the body cavity; and
exposing a portion of each of the plurality of meshes with optical energy, such that the plurality of surgical meshes adhere to one another.

2. A method in accordance with claim 1, wherein each of the plurality of coatings includes collagen.

3. A method in accordance with claim 2, wherein the collagen is derivatized with a carboxyl group.

4. A method in accordance with claim 1, wherein the chromophore is riboflavin.

5. A method in accordance with claim 1, wherein the chromophore is selected from a group consisting of lumiflavin and lumichrome.

6. A method in accordance with claim 1, wherein the optical energy is light having a wavelength in a range of 365 nm to 375 nm.

7. A method in accordance with claim 1, wherein the optical energy is light having a wavelength in a range of 440 nm to 480 nm.

8. A method in accordance with claim 1, wherein the material is provided in a plurality of tacks in each of the plurality of surgical meshes, the method further including:
aligning a first one of the plurality of tacks in a first one of the plurality of surgical meshes with a second one of the plurality of tacks in a second one of the plurality of surgical meshes, such that the first one of the plurality of tacks overlaps and contacts the second one of the plurality of tacks,
wherein said exposing includes exposing the first and second ones of the plurality of tacks.

9. A method in accordance with claim 1, wherein the material is provided as a coating of each of the plurality of surgical meshes, the method further including:
overlapping a first portion of a first one of the plurality of surgical meshes with a second portion of a second one of the plurality of surgical meshes,
wherein said exposing includes exposing the first portion of the first one of the plurality of surgical meshes and the second portion of the second one of the plurality of surgical meshes.

10. A method, comprising:
providing a plurality of surgical meshes, each of the plurality of surgical meshes including a material that has a chromophore; and
exposing a portion of each of the plurality of meshes with optical energy, such that the plurality of surgical meshes adhere to one another.

11. A method in accordance with claim 10, wherein each of the plurality of coatings includes collagen.

12. A method in accordance with claim 11, wherein the collagen is derivatized with a carboxyl group.

13. A method in accordance with claim 10, wherein the chromophore is riboflavin.

14. A method in accordance with claim 10, wherein the chromophore is selected from a group consisting of lumiflavin and lumichrome.

15. A method in accordance with claim 10, wherein the optical energy is light having a wavelength in a range of 365 nm to 375 nm.

16. A method in accordance with claim 10, wherein the optical energy is light having a wavelength in a range of 440 nm to 480 nm.

17. A method in accordance with claim 10, wherein the material is provided in a plurality of tacks in each of the plurality of surgical meshes, the method further including:
aligning a first one of the plurality of tacks in a first one of the plurality of surgical meshes with a second one of the plurality of tacks in a second one of the plurality of surgical meshes, such that the first one of the plurality of tacks overlaps and contacts the second one of the plurality of tacks,
wherein said exposing includes exposing the first and second ones of the plurality of tacks.

18. A method in accordance with claim 10, wherein the material is provided as a coating of each of the plurality of surgical meshes, the method further including:
overlapping a first portion of a first one of the plurality of surgical meshes with a second portion of a second one of the plurality of surgical meshes,
wherein said exposing includes exposing the first portion of the first one of the plurality of surgical meshes and the second portion of the second one of the plurality of surgical meshes.

19. A method in accordance with claim 10, further including:
inserting the plurality of surgical meshes into a body cavity.

20. A method in accordance with claim 19, further including:
providing the plurality of meshes on a wound site in the body cavity.
